(19) United States
(12) Patent Application Publication
(10) Pub. No.: US 2008/0300277 A1
(43) Pub. Date: Dec. 4, 2008

(54) PROCESS TO IMPROVE STABILITY OF A PHARMACEUTICAL COMPOSITION

(76) Inventors: Andrea Fetz, Wetzikon (CH); Georg Ludwig Kis, Triboltingen (CH); Michel Pepiot, Annonay (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/016,428

(22) Filed: Jan. 18, 2008

Related U.S. Application Data
(63) Continuation of application No. 10/451,088, filed on Jun. 17, 2003, now abandoned.

(30) Foreign Application Priority Data
Dec. 22, 2000 (EP) 00128318.3

(51) Int. Cl. A61K 31/4535; A61P 27/02

(52) U.S. Cl. 514/324

(57) ABSTRACT

The present invention describes in particular a method for stabilizing a pharmaceutical composition by contacting said composition with a polymeric material comprising in particular an ethylene oxide sterilization step.

The present invention describes in particular a method to improve the stability of a pharmaceutical composition by contacting said composition with a polymeric material comprising in particular an ethylene oxide sterilization step.

Pharmaceutical compositions, in particular aqueous pharmaceutical compositions are typically provided in containers, which containers must be sterilized before filling. A problem arises if a container comprises a squeezable material such as polyethylene (PE), polypropylene (PP) and/or polyethylene terephthalate (PET) because these materials may for example not be treated with heat, because these may melt. Alternative sterilization treatments are known in the prior art and is for example ethylene oxide (ETO) treatment or gamma irradiation treatment.

We have found that the stability of an aqueous pharmaceutical composition is typically unacceptable if filled into PE containers which have been previously sterilized by gamma irradiation treatment as known and practiced in the prior art.

Further we have found that the problem may be solved if the sterilization of an empty PE, PP and/or PET container is carried out with ETO e.g. as known and practiced in the prior art before filling said empty container with an aqueous pharmaceutical, composition.

The present invention therefore relates in particular to the use of an ETO sterilized PE, PP and/or PET container to improve the stability of an aqueous pharmaceutical composition, in particular to improve the stability of a composition being susceptible to oxidative degradation.

As used herein, ETO sterilized, refers in particular to the treatment steps of:

Exposing a container, in particular an empty PE, PP and/or PET container, to ethylene oxide (ETO) at room temperature, at a concentration and for a time sufficient to achieve sterility; and thereupon, removing said ETO under aseptic conditions from said container for a period sufficient to achieve an ETO content of less than 1 ppm.

Therefore, an ETO sterilized container is typically a container, which has been subjected to said treatment steps.

The following parameters are preferably applicable for said ETO sterilization procedure:

The ETO concentration is typically characterized by its composition, namely it contains for example 25% (vol./vol. at room temperature) nitrogen, more preferably 50% and in particular 75% nitrogen and/or carbon dioxide.

The ETO exposure time sufficient to achieve sterility is generally carried out for a time of 0.5-24 hrs, preferably 2-15 hrs and more preferably for a period of 3-12 hrs.

The ETO removal time, for a period sufficient to achieve an ETO content of less than 1 ppm, is typically for a period of 1-20 days, preferably 5-15 days, and more preferably for 8-10 days.

Removing of said ETO is typically carried out by air diffusion and/or by flushing said container aseptically with a gas selected from nitrogen, argon, carbon dioxide, air and preferably with nitrogen.

The present invention further relates to a method to improve the stability of a pharmaceutical composition which is sensitive towards oxidation, comprising the steps of:
exposing a squeezable container, in particular an empty PE, PP and/or PET container, to ethylene oxide (ETO) at room temperature, at a concentration and for a time sufficient to achieve sterility,
removing said ETO under aseptic conditions from said container for a period sufficient to achieve an ETO content of less than 1 ppm,
transferring under aseptic conditions a pharmaceutical composition into said sterilized container, and
closing said container comprising said pharmaceutical composition with a closing device.

The above method steps are typically carried out in a conventional manner or in an analogous manner to that described in the examples or in a manner as described in the examples.

In the context with the present invention the preferred embodiments are described above and below.

As used herein, stabilization relates to the stability of the pharmaceutical composition in total and in particular to the stability of the active ingredient itself when exposed to storage (shelf life stability).

The term squeezable material relates preferably to a plastic material and in particular to low density polyethylene (LDPE), high density PE (HDPE), polypropylene (PP), (PET) and mixtures thereof. A preferred material is LDPE and HDPE, even more preferred is LDPE.

The term container relates preferably to a bottle, in particular to a bottle as used for providing liquid aqueous pharmaceutical compositions. A highly preferred container is a bottle comprising LDPE.

Consequently, the term container relates in particular to a polyethylene bottle and in particular to a LDPE bottle. Such bottles may optionally contain further auxiliaries such as a light absorbing material e.g. titanium dioxide, a color pigment, a UV-absorber, an antioxidant and/or the like.

As used herein, the LDPE material typically contains no antioxidant, however HDPE may contain an antioxidant such as e.g. butylhydroxytoluene (BHT). In an example, a bottle is manufactured from LDPE containing no antioxidant, its cap from HDPE containing BHT.

A pharmaceutical active compound is e.g. selected from the group of compounds which act for example as:

Anti-Inflammatory drugs, such as steroids, e.g. dexamethasone, fluorometholone, hydrocortisone, prednisolone; or so-called non-steroidal anti-inflammatory drugs (NSAID) such as COX-inhibitors, e.g. diclofenac, ketorolac, or indomethacin;
Antiallergic drugs, selected e.g. from cromolyn, ketotifen, levocabastine, olopatadine, and rizabene,
Drugs to treat glaucoma (in particular intraocular pressure treatment), selected e.g. from latanoprost, 15-keto-latanoprost, unoprostone isopropyl, betaxolol, clonidine, levobunolol and timolol;
Anti-infective drugs, e.g. selected from chloramphenicol, chlortetracycline, gentamycin, neomycin, ofloxacin, polymyxin B and tobramycin;
Antifungal drugs, e.g. selected from amphotericin B, fluconazole and natamycin;
Anti-viral drugs such as acyclovir, fomivirsen, ganciclovir, and trifluridine;
Anesthetic drugs, e.g. selected from cocaine hydrochloride, lidocaine and tetracaine hydrochloride;
Miotics, e.g. selected from carbachol, pilocarpine and physostigmine;
Carbonic anhydrase inhibitors, e.g. selected from acetazolamide and dorzolamide;
Alpha blocking agents, e.g. selected from apraclonidine and brimonidine; and
Antioxidants and/or vitamins, e.g. selected from retinol, retinol acetate, and retinol palmitate.

Preferred pharmaceutically active compounds are selected from the group of anti-inflammatory drugs, antiallergic drugs and drugs to treat glaucoma.

Other preferred pharmaceutically active compounds are selected from the group of diclofenac, 15-keto-latanoprost, ketorolac, ketotifen, latanoprost, levobunolol, levocabastine, ofloxacin, pilocarpine, polymyxin B, prednisolone, retinoic acid, retinol, retinol acetate, retinol palmitate, tetracycline, unoprostone isopropyl, and pharmaceutically acceptable salts thereof.

More preferred pharmaceutically active compounds are selected from the group of, betaxolol, chloramphenicol, diclofenac, ketotifen, levobunolol, levocabastine, pilocarpine, retinoic acid, retinol, retinol acetate, retinol palmitate, unoprostone isopropyl, and pharmaceutically acceptable salts thereof.

Highly preferred is ketotifen, retinoic acid, retinol, retinol acetate, retinol palmitate, unoprostone isopropyl, and pharmaceutically acceptable salts thereof.

Very particular preferred is ketotifen and pharmaceutically acceptable salts thereof, e.g. the hydrogen fumarate (hereinafter tills salt is often referred to as Compound A).

As used herein, a pharmaceutical composition is characterized by the carrier wherein said pharmaceutical active compound is mixed, suspended, dissolved and/or partially dissolved. Such a carrier may be chosen e.g. from a wide variety of carriers used preferably for ophthalmic compositions. It may be based on a solvent selected from the group consisting of water, mixtures of water and water-miscible solvents, such as C₁- to C₇-alkanols, e.g in the case of compound A glycerol A highly preferred carrier is water. The concentration of the carrier is, typically, from 1 to 100000 times the concentration of the active ingredient. The term aqueous typically denotes an aqueous composition wherein the carrier is to an extent of >50%, more preferably >75% and in particular >90% by weight water.

A preferred pharmaceutical composition is preferably adapted to ophthalmic prerequisites (e.g. ocular compatibility) and is in particular an ophthalmic composition.

For Compound A typical concentrations are:

i) 0.025%
ii) 0.05%

Further preference is given to a pharmaceutical composition which is suitable for ocular administration. Therefore such a pharmaceutical composition preferably comprises further ingredients in order to meet the prerequisites for ocular tolerability.

In a particular aspect, the present invention relates to the stabilization of an ophthalmic composition and in particular to an aqueous ophthalmic composition.

Further aspects of the present invention are those disclosed in all dependent and independent claims.

A further aspect of the present invention is the use of a LDPE bottle, which has been subjected to ETO exposure e.g. in accordance to the working examples of the present application, for improving the stability in particular towards oxidation of an ophthalmic composition, e.g. a ketotifen 0.025% solution, which composition is subsequently transferred into said bottle in accordance to the disclosure of the present invention.

As used herein % refers to weight/weight (W/W) if not specified differently.

The pharmaceutical compositions of the present invention may be used for the known indications of the pharmacologically active agent.

In a further aspect the present invention provides a container containing a sterile pharmaceutical composition, which container has been ETO sterilized and is obtainable by a method or process as described above,

a) wherein the active is other than ketotifen
b) contains ketotifen and is produced other than a process as described in an example.

In yet a further aspect the present invention provides an unclosed ETO sterilized container containing a sterile pharmaceutical composition.

In yet another aspect the present invention provides an unclosed container treated by ETO as described herein containing a sterile pharmaceutical composition, in particular a ketotifen composition.

The closing device of an above described container may be manufactured from PE, PP and/or PET, such as HDPE, and might still be sterilized by gamma irradiation, in particular if said closing device will—to a substantial degree—not contact an above pharmaceutical composition.

### EXAMPLE 1

### Ophthalmic Eye Drop Composition Comprising Ketotifen

The manufacture of the ophthalmic solution is described for a typical example. All the ingredients are dissolved in water for injections and the pH of the solution is adjusted. The solution is then brought to the final weight and sterile-filtered into a bulk container which is then used to fill the product into sterilized containers. Manufacture is carried out according to GMP guidelines.

The solution is filed into pre-sterilized bottles and plugged and capped with sterile components within a sterile environment using aseptic techniques.

Development studies showed that steam sterilization (i.e. terminal sterilization) was not acceptable due to heat-sensitivity of product and container (PE-bottle). Sterilization by filtration with subsequent aseptic filling into sterile containers is standard industry practice for ophthalmic solutions.

The bulk solution is routinely assessed for bioburden prior to sterile filtration and the EU limit of 10 organisms per 100 ml is adhered to. The sterilizing grade membrane filters are tested for integrity and checks on pH, osmolality, odor and physical appearance provide suitable in-process controls.

A ketotifen eye drop solution comprises e.g.:

| Composition | |
|---|---|
| ketotifen hydrogen fumarate | 0.0345% |
| (ketotifen content) | (0.025%) |
| glycerol, pure compound | 2.125% |
| benzalkonium chloride | 0.01% |
| sodium hydroxide 1N | 0.074% |
| water for injection ad | 100 ml |
| pH | 5.32 |
| Osmolalilty (mOsmol) | 240 |

### EXAMPLE 2

The stability of the example 1 composition is investigated for their shelf stability in containers (or packaging components) being sterilized with different methods of sterilization.

The packaging components of Ketotifen 0.025% Eye Drops are sterilized by gamma irradiation with a minimum dosage of 25 kGy (sample III). Six batches of 10 to 400 litres are made for stability testing.

The release results from these batches are satisfactory with no significant variation between batches. However, the results of stability tests show significant differences. While some batches remain stable for a longer time, others show a significant decrease of the active compound ketotifen fumarate already within months. It is presently assumed that this phenomenon may be related to the gamma irradiation of the bottles. Therefore, an accelerated stability study is carried out to test this hypothesis. Ketotifen 0.025% Eye Drop solution is filled into untreated bottles, gamma irradiated bottles and bottles sterilized by ethylene oxide and all the samples are stored at 80° C. for 15 hours. The test results are compared in the table reproduced infra:

Based on these data, it is observed that sterilization of the LDPE bottles and droppers by ethylene oxide is a superior treatment for Ketotifen 0.025% Eye Drops. It should be emphasized that the containers will only be used when residual ethylene oxide has fallen below the 1 ppm level (e.g. ventilation of the containers for about two weeks after ETO exposure (treatment)). The HDPE closures might still be sterilized by gamma irradiation since they are not in contact with the eye drops.

| Sample | I | II | III | IV | V |
|---|---|---|---|---|---|
| 0-value: | | | | | |
| pH | 5.28 | 5.28 | 5.25 | 5.40 | 5.42 |
| Osmolality (mOsmol) | 238 | 238 | 240 | 241 | 244 |
| % ketotifen | 100.2 | 100.2 | 99.8 | 99.8 | 102.4 |
| % degradation product I | n.d. | n.d. | n.t. | n.d. | n.d. |
| % degradation product II | n.d. | n.d. | 0.05 | n.d. | n.d. |
| stress test at 80° C., | | | | | |
| 15 hours: | | | | | |
| pH | 5.2 | 4.83 | 4.75 | 5.22 | 5.24 |
| Osmolailty (mOsmol) | 241 | 244 | 241 | 241 | 248 |
| % ketotifen | 96.8 | 91.6 | 88.6 | 94.5 | 97.7 |
| % degradation product I | ~0.05 | ~1.4 | 1.2 | n.d. | n.d. |
| % degradation product II | ~0.1 | ~3.2 | 2.8 | n.d. | n.d. |

Legend:
Sample I: Freshly prepared eye drops filled in untreated PE bottles.
Sample II: Freshly prepared eye drops filled in gamma irradiated (40 kGy) PE bottles.
Sample III: Freshly prepared eye drops being subsequently stored at 5° C. for several days, filled in gamma irradiated (at least 25 kGy) PE bottles.
Sample IV: Freshly prepared eye drops aseptically filled in ETO sterilized PE bottles.
Sample V: Repetition of IV.
Degradation product I and II respectively denote ketotifen N-oxide which is an oxidation product of ketotifen. It exists in form of two diastereomers with the same stoichiometric formula.
% denotes total weight %
n.d. means: not detectable; below limit of detection
n.t. means: not determinable; above limit of detection, but below limit of quantitation

The HPLC method has been shown to be selective for ketotifen hydrogen fumarate as well as to all the following known impurities which might possibly be found in the eye drops as follows:

### Shelf Life Stability:

The finished product, ketotifen 0.025% eye drops stored in ETO sterilized PE containers, exhibit an improved stability compared with that of ketotifen 0.025% eye drops stored in gamma irradiated PE containers (sample III). The results demonstrate the good stability of ketotifen 0.025% eye drops for 12 months when stored at temperatures up to 25° C.

### CONCLUSION

Sterilization of the containers by ethylene oxide is the method of choice since gamma irradiation was shown to be detrimental to the stability of the solution.

1. Method to improve the stability of a pharmaceutical composition which is sensitive towards oxidation, comprising the steps of:
exposing an empty PE, PP and/or PET container to ethylene oxide (ETO) at room temperature, at a concentration and for a time sufficient to achieve sterility,
removing said ETO from said container under aseptic conditions for a period sufficient to achieve an ETO content of less than 1 ppm,
transferring under aseptic conditions a pharmaceutical composition into said sterilized container, and
closing said container comprising said pharmaceutical composition with a dosing device.

2. Method of claim 1, wherein said pharmaceutical composition is an aqueous ophthalmic composition.

3. Method of claim 1, wherein said container is a LDPE and/or HDPE container, more preferably a LDPE container, and in particular a LDPE container.

4. Method of claim 1, wherein said pharmaceutical composition comprises a pharmaceutically active ingredient selected from the group consisting of diclofenac, 15-keto-latanoprost, ketorolac, ketotifen, latanoprost, levobunolol, levocabastine, ofloxacin, pilocarpine, polymyxin B, prednisolone, retinoic acid, retinol, retinol acetate, retinol palmitate, tetracycline, unoprostone isopropyl, and pharmaceutically acceptable salts thereof.

5. Method of claim 1, wherein said ETO is removed air diffusion and/or by flushing said container aseptically with a gas selected from nitrogen, argon, carbon dioxide, air and preferably with nitrogen.

6. Method of claim 1 or 6, wherein said ETO is removed for a period of 1-20 days, preferably 5-15 days, and more preferably for 8-10 days.

7. Method of claim 1, wherein said container is exposed to ETO for a period of 0.5-24 hrs, preferably 2-15 hrs and more preferably for a period of 3-12 hrs.

8. Method of claim 1, wherein said ETO contains 25% (vol./vol. at room temperature) nitrogen, more preferably 50% and in particular 75% nitrogen and/or carbon dioxide.

9. Method of claim 1, wherein the closing device is sterilized via gamma irradiation.

10. A process for the production of a stable pharmaceutical composition in a container, comprising the steps of:
a) sterilizing a container, in particular a PE and/or PP container, with ethylene oxide (ETO) at room temperature, at a concentration and for a time sufficient to achieve sterility,
b) removing said ETO from said container under aseptic conditions for a period sufficient to achieve an ETO content of less than 1 ppm, for example under air diffusion conditions,
c) transferring under aseptic conditions a pharmaceutical composition into said sterilized container, and
d) closing said container comprising said pharmaceutical composition with a closing device.

11. A process of claim 10, wherein said closing device is sterilized via gamma irradiation.

12. Use of an ETO (ethylene oxide) sterilized PE, PP and/or PET container to improve the stability of an aqueous pharmaceutical composition, in particular to improve the stability of a composition being susceptible to oxidative degradation.
